(19) United States
(12) Patent Application Publication
(10) Pub. No.: US 2023/0410681 A1
(43) Pub. Date: Dec. 21, 2023

(54) Apparatus and Method for Gamification of Nutrition and Food Education for Young Children

(71) Applicants: Emillia Jarvinen, Espoo (FI); Tuulia Jarvinen, Espoo (FI)

(72) Inventors: Emillia Jarvinen, Espoo (FI); Tuulia Jarvinen, Espoo (FI)

(21) Appl. No.: 17/752,890

(22) Filed: May 25, 2022

(51) Int. Cl. G09B 19/00; G16H 20/60

(52) U.S. Cl. CPC G09B 19/0092; G16H 20/60

(57) ABSTRACT

An application, system and a method for creating a gaming experience for families and promotes the well-being and public health, combining eating vegetables with the features of the game, both at home and at the groceries. The application, system and a method combines modern technologies that interest the child and creates tools for the guardian to inspire the child to eat more variety of vegetables, fruits, nuts and berries.

### BACKGROUND

### Field of the Invention

Embodiments of the present invention relate generally to nutrition education for children, and more particularly to an apparatus and method for combining nutritional education with interactive elements in engaging ways in grocery stores, retail stores, cafeterias or equivalent locations for young children in order to affect the dietary habits of young children.

### Description of the Prior Art

According to the studies, for instance at https://medcraveonline.com/AOWMC/fruit-and-vegetablesrsquo-consumption-among-children-and-adolescents-determinants-of-consumption-and-possible-solutions.html, low consumption of vegetables, fruits, nuts and berries in peoples' eating habits is a great problem for the public health and especially for children. The problem is highlighted when children grow and their eating habits follow them into adulthood. According to the latest studies, https://www.jandonline.org/article/S2212-2672(21)00414-7/fulltext #relatedArticles, only 1% of the young Finnish children (of 2-6 years) consume sufficient amount of vegetables, fruits, nuts and berries daily and in many cases children's sugar intake often exceeds the recommended dosage from the age of two onwards.

Because of this, the number of overweight or obese children is on the rise, as are health challenges linked with overweight and obesity. Often these health challenges follow individuals from childhood to adulthood.

Parents often lack effective tools to promote healthy eating habits, especially for promoting the consumption of versatile vegetables, fruits, nuts and berries. Instead, there is a lot of promotion towards consumption of unhealthy food.

Until this date, various attempts to fix this problem have been made. There has been a number of educational campaigns at school restaurants, cafeterias or eating areas. Nutritional education has been offered at maternity clinics, kindergartens, libraries and at schools.

Other attempts have been made to introduce the child to vegetables in early childhood education, schools and similar locations. These introductions have been made for example by letting children taste different food stuffs, or with Sapere method, in which children learn new things about food using the senses of smell, taste, touch, vision and hearing.

Producers of vegetables or fruits, nuts or berries have given lectures at schools. Other attempts have included providing children with cooking courses (online, group or mobile) or with different kind of games (mobile games, board games) for them in order to educate their selves about healthier eating habits. Lately, a selection of healthy food stuffs for children have been introduced and marketed. These foodstuffs include for example baby carrots, smoothies with more vegetables, snack bars and delicacies made mainly from vegetables, fruits, nuts or berries.

The attempts mentioned here might have helped children individually, but the problem at large is still unremedied and could worsen in the near future, according to the latest studies https://www.thelancet.com/journals/landia/article/PIIS2213-8587(21)00314-4/fulltext.

There is a need for an intervention which leads into motivation and habit change straight from the early years and the societies are still lacking effective tools.

There have been efforts that would motivate children and change their eating habits from the early years.

Publication US2011/0300518A1 discloses a nutritional board game which educates children about advantages of a nutritionally-balanced diet.

In one aspect, the nutritional game includes a board having an illustration of a character, such as a lovable bear character, in which the board is divided into a plurality of sectors configured to be covered by one of a plurality of nutritional cards to begin the game. As the child eats various foods over the course of the day, the child removes one or more of the nutritional cards from the board representing the foods that child has eaten until all of the nutritional cards on the board are removed, thereby exposing the lovable character on the board to the child.

The invention disclosed in the patent application encourages children to eat healthy food, but it fails to take into consideration many things that might have an impact on eating habits of an individual child, such as possible allergies of a child, seasonal & local offerings of fruits, vegetables and berries at which time the seasonal products are often more reasonably priced. The invention does not provide food educational tools for the parents to help the children eat healthier, nor recipes.”

The disclosed invention utilises nutritional recommendations, which were in force at time when the board game was produced. As science evolves, nutritional recommendations also change over time, and the disclosed invention fails to do so.

In order to avoid child obesity and various illnesses that come with obesity and promote healthier nutrition and public health in general, there is need for an immersive gaming application that gathers data from a variety of sources and encourages the user regularly to eat healthy and follow diet that meets up-to-date health recommendations, seasonal offerings of vegetables, fruits, nuts and berries and is allergen-free for the user.

To solve the problems mentioned above, we have developed a system, an arrangement and a method for gamification of nutrition and food education for young children and their guardians.

### SUMMARY OF THE INVENTION

An object of the present invention is to provide a gamification of nutrition education for young children arrangement, method for gamification of nutrition education for young children and computer program product in order to promote healthier eating habits by increasing vegetable, fruit and berries consumption and promote public health.

The object of the invention is achieved by a method, an arrangement and computer program product which are characterized by what is stated in the independent claims. Some embodiments of the disclosure are disclosed in the dependent claims.

The present invention brings vegetables, fruits, nuts and berries into application which turns eating sufficiently vegetables, fruits, nuts and berries into a game. The application is used in children's or guardian's mobile terminal and turns education about healthier eating choices into a game experience using image recognition, grocery stores' back-end systems, anonymized user information, AI and augmented reality.

The present invention discusses vegetables, fruits, nuts and berries, but it is obvious to a person skilled in the art that the disclosed system, arrangement and method can be utilized in promoting nutritional eating habits in general, drinking sufficiently water, taking medicine regularly etc.

In the future, the disclosed system, arrangement and method can be developed to meet other health challenges, including type 2 diabetes, by increasing understanding of, for example, the glycemic index and load of a foodstuffs.

The application rewards children in various ways for completed challenges, and provides parents and children with recommendations and knowledge about nutritional facts of specific vegetables, fruits, nuts and berries, and about health benefits of consumption of sufficient number of vegetables, fruits, nuts and berries.

The apparatus includes an application to be installed on a mobile device and its backend system, wherein the application introduces its user to a character named Vegemi. Vegemi is a character in the app that is a good intestinal bacterium who needs vegetables, fruits, nuts and berries on a regular basis to conserve its energy, and guides users to take photographs of all the vegetables, fruits, nuts and berries they consume and encourages users to consume more vegetables, fruits, nuts and berries.

The character's visual representation changes either momentarily by showing the user an animation or for a longer period of time by giving the user's character a sticker or similar icon when the user photographs the vegetables, fruits, and berries they have eaten. The animations shown are presented in a way that fascinates the user, typically the child, and further encourages the eating of vegetables, which at the same time creates an understanding of the relevance of vegetables to well-being. For example, a user could see an animation of improved vision after shooting a carrot, and could get a sticker or icon depicting a carrot next to their game character. The animations to be shown are selected on the basis of health claims made by health authorities, such as the EFSA (European Food Safety Authority), FDA or other authorities' health claims.

For example, when a user photographs a carrot within the application, the application recognizes it as a carrot using an external image recognition service, and performs an animation associated with the carrot. For example, animation could be related to the health effects of a carrot, such as maintaining a sense of sight. In this case, for example, the character could get glasses or binoculars to see better.

Alternatively, to photographing vegetables, fruits, nuts or berries before eating them, the user might take photograph of banana peels, apple seed pot or other left over of vegetables, fruits, nuts or berries. The application recognizes it similar way and provides user with similar effect.

The character also gets a sticker or similar icon on the screen after eating the required amount of vegetables, fruits, nuts or berries. The required amount can be set for the user on a per-user basis, or based on user-defined activity. When the application is downloaded and installed on a mobile device, these settings can be set upon first launch of the application. The settings can be set at later time or adjusted when needed. Typically, the application is downloaded and installed by a child's guardian who determines the required amounts of vegetables, berries, or fruits. Alternatively, the guardian accepts, by default, initial values selected in accordance with the nutritional recommendations of the authorities and encouraging children to eat a wide range of vegetables, fruits, nuts and berries. At this time, the guardian also notifies the application about possible allergies the user might have, so that the application doesn't recommend a vegetable, fruit or berry to user that the user is allergic to.

If the user does not continue to eat and photograph the required amount of that vegetable, fruit or berry in set time frame the sticker or similar icon will also be lost. This indicates that the user has not recently eaten the required amount of that vegetable, fruit or berry. This feature of the application is intended to encourage users to regularly consume vegetables, fruits, nuts or berries and photograph them in the app. The application will in this case remind, inspire & encourage the guardian to continue using the application and healthy diet.

Alternatively, to photographing vegetables, fruits, nuts or berries, the user can interact with materials placed in the vegetable department of the store, for example, the user can scan a barcode or QR code from a poster installed in the vegetable department on their mobile device, read an NFC or RFID tag on their mobile device, identify a Bluetooth beacon after which the application reacts in a similar way as when the user photographs the vegetables, fruits, and berries he has eaten. This allows stores to enhance the consumer demand for certain seasonal vegetables, fruits, nuts or berries, for example, or if there is a large supply of that plant, for example due to a good harvest or great climate impact or novel veggies or fruits, nuts and how to use them. The partner retailer can also send targeted bargains of chosen vegetables for the users of the application, ensuring there is demand to meet the local supply and knowledge to use the local fresh produce.

Alternatively, to photographing vegetables, fruits, nuts or berries, the user or user's guardian, such as his/her parent or teacher can enter information into application about consumed vegetables, fruits, nuts or berries.

A user interaction with vegetables, fruits, and berries, in the context of this disclosure, refers to an event in the application which acts as an event similar to eating of the vegetables, fruits, and berries.

A user photographing vegetables, fruits, and berries in the application, in the context of this disclosure, refers to an event in the application which acts as an event similar to eating of the vegetables, fruits, and berries.

The application rewards the user's perseverance by providing visual changes to the user's character if the user has regularly photographed the vegetables, fruits, or berries they have ingested or interacted with systems installed in stores. Rewarding takes place with changes in the visuality of the character that reflect the user's progress in the application. Such visual changes may include, for example, changes in the character's size, colour, by including smaller buddy characters available to the character, character accessories, or combinations thereof. Additionally, the application can provide the character with an additional image of consumed vegetables, fruits, or berries with a number of consumed vegetables, fruits, or berries.

The application can send reminders or other messages to the user or his/her guardian(s) encouraging the user to eat more vegetables, fruits, and berries. Reminders can appear in the app's message window, in the application, as push messages to your mobile device, or in other similar ways. Messages can be text, images, sounds, speech, animations, or combinations thereof.

Reminders or other messages may relate to, for example, fruit recommended to the user if, for example, it appears that the user has consumed enough vitamins A and fibres but the user has not taken enough vitamin C. In this example, the message to the user may be, for example, a suggestion to enjoy an orange, or if the information in the application indicates that the user is a small child who is unlikely to read, a picture of an orange, an animation of an orange, or a similar message.

Reminders or other messages may be sent also in case of excessive consumption of certain vitamin, trace element or fibre, or the intake of vitamins differ greatly from the recommendations.

The application can take into account the vitamin or trace element needs of the user and compare them with the vegetables, fruits, nuts or berries photographed or interacted by the user. If the application detects a deficiency in a particular vitamin, trace element or fibres, the application may remind the user of a vegetable, fruit or berry that contains that vitamin, fibre or trace element. The application takes into consideration possible allergies of the user, and only suggests suitable vegetables, fruits, nuts or berries to the user.

The application may further combine information about the vegetables, fruits, nuts or berries photographed or interacted by the user with other information that may be in the current harvest season, for example, when vegetables, fruits, nuts and berries are generally cheaper to purchase. Other information can be the user's location/city/country, the user's age, or similar parameters obtained from the user and known to the application.

The application asks for information about its user during installation and first start-up, so that the application can better customize the experience for its user. The information requested by the application includes, for example, information about the user's vegetables, fruits, nuts and berries consumption, age, gender, health, weight, height, allergies, activity using the application, the user's country or city of residence, or some of the above.

The data or part of the data about user may be required to be filled in before the application starts, alternatively the data entry is optional. The information can be updated later if the user so wishes. The application takes into account information about the user's usage of the app to make the gaming experience enjoyable for those who use the application on a daily basis as well as for those who only use the application once or twice a week. In this case, for example, the amounts of berries, fruits, nuts or vegetables photographed or interacted with that affect the game character's progress are different for users with different usage activity.

The application also takes into account the user's possible allergies, recommending the replacement of another vegetable, fruit or berry that contains similar amounts of vitamins and trace elements, but does not cause allergic reactions in the user.

During the start-up, the application asks also to set optional goals for the user, the user can add them independently of variety of choices, or the application may also suggest the goals. The suggestions are based by combining the information requested by the user, overall user data and official health and nutritional databases. The goals are mainly targeted to support parents' guidance. Goals are achieved also by playing the game. If the goals are not achieved, the application will recommend actions (with notifications/emails) or ask to modify the goals. If the goals are achieved, the user will get rewards (such as mentioned before—a new feature or modifications on a figure or its surroundings, and the guardian is asked to set up new goals for the user. Alternatively, the application can recommend new goals for the user.

The application can be used as an individual user, in which case, for example, a parent downloads the application for the use of their child. The application can also be used for a group of several children. In this case, for example, a kindergarten teacher can download the application to his/her phone and create separate profiles for the children in their group. The teacher then can present users with challenges, for instance asking users to eat five carrots within a week. The teacher or parent can also use application to mark a certain vegetable, fruit or berry consumed by the group or single user. This feature is beneficial for instance, if there is a snack within kindergarten, where each member of a group is given a vegetable, fruit or berry to eat.

The teacher or parents of user can also set nutritional goals for the user or the group.

The application also includes a reporting section where guardians can see their child's or group of children's activity, as well as a summary of the nutrients generated by the application based on official open sources such as Fineli (Fineli is a national food composition database of Finland, https://www.suomi.fi/services/eservice/fineli-finnish-food-composition-database-the-national-institute-for-health-and-welfare-thl/ba003f23-14e6-46b9-985c-30d654e35ef2) and makes recommendations based on missing nutrients, harvest seasons and possible allergies of the user.

For example, if a child has photographed only cucumbers, the application will tell what nutrients the user has received, and will recommend vegetables to add to the diet based on nutrient recommendations, taking into account the region-specific harvest season of vegetables and the user's allergies.

Certain goals can also be set for the user or a group of users, and the application can reward the user or a group of users for achieving those goals.

The app may also include a variety of mini-games to help the user learn more about vegetables, fruits, or berries. The user can e.g. calls for different vegetables to be combined with different health benefits so that children learn about the health benefits. Health benefits can be described for example in writing, by image, by animation, by speech, by sound, or a combination of these. The user can get riddles about the nutrients and properties of vegetables, fruits, nuts and berries, such as the example; which vegetable provides the brain with important fatty acids, while increasing the user's understanding of the link between food and well-being. From successful games, the character gains strength, feels good and looks more cheerful, and is rewarded with various decorations, which will also be emptied after the prescribed time has elapsed. Through games, the user also learns about the origin of vegetables, fruits, nuts and vegetables and where they grow, for example by combining the right place to grow (tree, vine, field, vegetable garden) with the right plant.

In addition, the application may include various virtual reality (VR/AR/MR/XR etc) functions, in which case the character of the application may be seen for example on a store's vegetable shelf, encouraging the user to buy products that are in season, for example.

By turning healthy eating into an immersive playing experiment with interacting with vegetables, fruits, nuts and berries and teaching the health benefits of vegetables, fruits, nuts and berries to both children and their guardians, we can improve children's eating habits and increase the amount of vitamins, fibres and trace elements in their diet. This contributes to reducing obesity in children and improving public health. These features will be explained in the following with the aid of the figures and their explanatory part.

The present invention relates to a method executable on a computer that encourages a child user to eat more vegetables, fruits, nuts or berries that includes placing an animated character on a computer screen representing a friendly intestinal bacterium, where the animated character has an energy level represented by a energy bars. The application decreases the energy level with time if the animated character is not fed at least one of a vegetable, a fruit, a type of nut, or a type of berry The application also causes the animated character to display unhappiness. The screen then displays a selection of icons representing vegetables, fruits, nuts and berries that the child can choose from to feed the animated character. The chosen vegetable, fruit, type of nut or type of berry can be emphasized as a reward to the child. An animated display of a caricature of the human body and the digestive track can be displayed to show that the animated character is being fed healthy food. Finally, the energy levels increase, and the animated character displays happiness.

### DESCRIPTION OF THE FIGURES

In the following, embodiments of the invention will be described with reference to non-limiting examples shown in the accompanying drawings, in which:

FIG. 1 is a block diagram of an embodiment of the present invention.

FIGS. 2-4 disclose examples of the user interfaces on an application installed on a mobile terminal

### DESCRIPTION OF THE PREFERRED EMBODIMENTS

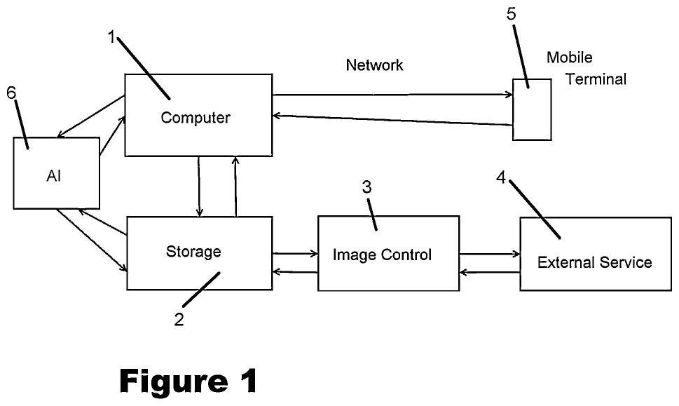

Turning to FIG. 1, a block diagram of an embodiment of the present invention can be seen. The system consists of computer (1), storage (2), image control part (3), external service (4), mobile terminal (5) and artificial intelligence function (6).

The computer (1) herein can be a server computer or a computer cloud or plurality of computers arranged to interact with client application installed on mobile terminal (5).

The computer may comprise a programmable logic and/or programmable microprocessor, for example. The computer (1) performs background tasks and responds to service requests for the application, enabling the application to function. The computer (1) is also in connection with external information systems, such as national food composition database of Finland, Dietary Guidelines for Americans, and European Food Safety Authority for instance, to retrieve up-to-date nutritional recommendations for the system.

Storage (2) stores information on, or accessible to, computer (1), and the application installed on mobile terminal (5) and external sources. Mobile terminal (5) is connected to computer (1) via a network.

Storage (2) is used to store information for immediate use and act as storage for storing information about computer program or application or “app”, its' users and their application data —related information. It can also store temporarily or permanently information retrieved from external systems (not visible in FIG. 1) concerning nutritional information about vegetables, fruits, nuts or berries, seasonal information about vegetables, fruits, nuts or berries, official and/or unofficial nutritional recommendations and information about stores' point of sales—systems. Storage can include computer memory, Disk Storage, Cloud Storage or the like. Storage can also include databases, file systems, mass memories, random access memories, read-only memories, non-volatile memories, or computer storage implemented in other ways. Storage 2 might be located on remote location or on computer (1). Computer 1 may be a remote server.

Image control part (3) receives image data from mobile terminal (5) via computer program (1) and storage (2) and sends it to an external service (4) for image validation and recognition of vegetable, fruit or berry images might contain. Image control part (3) might be a computer program running on remote computer or part of data of computer (1).

External service (4) receives, processes and returns recognized images to image control part (3) with information on the recognized vegetable, fruit or berry. The information typically consists of the name of vegetable, fruit or berry and/or number of vegetables, fruits, nuts or berries in the image. The results are stored in storage (2) and the computer program resident on computer (1) instructs client application on mobile terminal (5) to function according to results, i.e., rewarding user with visual changes on the character or messaging user that the image was not recognized. The Mobile Terminal 5 is preferably a smartphone or other type of mobile phone. It can also be a tablet or laptop computer or any other type of mobile computer.

External service (4) could be produced and offered by Google, Amazon, IBM, Microsoft or other service provider.

Artificial intelligence (AI) (6) can be used within the system in order to improve recommendations of vegetables, fruits, nuts or berries to the user. Artificial intelligence 6 can retrieve data from storage 2 about vegetables, fruits, nuts or berries, their nutritional values including amounts of different vitamins, fibres and trace elements, seasonal information from harvest calendars in order to decide which vegetables, fruits, nuts or berries are in season and therefore inexpensive to buy. Artificial intelligence 6 then retrieves information storage (2) about the user. This information includes for example Allergies of the user, users' application usage history, users' favourite vegetables, fruits, nuts and berries, user's location/city/country, user's age, and anonymized user data from large set of users and their preferences and consumption/interaction histories on vegetables, fruits, nuts or berries. By consumption histories we mean information which vegetables, fruits, nuts or berries user has photographed, interacted with or played minigames with within the application.

Artificial intelligence (6) is taught or trained to identify vegetables, fruits, nuts or berries that the user has not consumed, but might be beneficial to user according to nutrition recommendations and anonymized user data from large set of users. Artificial intelligence 6 is programmed to identify and suggest new vegetables, fruits, nuts or berries user would enjoy. This is achieved by identifying peer groups, users with similar taste and if known, age group, location and users' consumption history.

Artificial Intelligence 6 can include one or more neural networks, trained to recognize images of vegetables, fruits, nuts and berries.

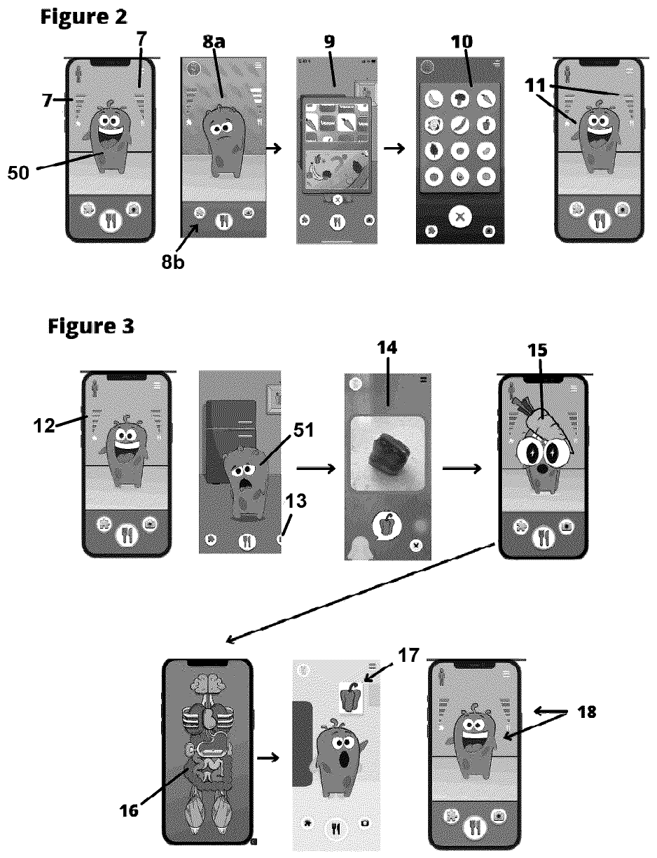

FIG. 2 depicts one example of the user interface of the application resident on user's mobile terminal 5.

The function of mini-games, such as puzzles, quizzes and the like in the application is to educate children about vegetables, fruits, nuts and/or berries. The education of children is encouraged by rewarding user in the game, as completing minigames increases character's energy levels. The bars show the energy level (7) of the character (50). When the energy level of the character decreases, character looks unhappy, as seen in (8a). The energy levels of character can be restored by playing minigames with the character. By pressing button (8b), the user can open the mini-games (9 & 10), and by playing minigames, user can increase the energy levels of the character and the character looks energized (11).

In addition to described minigames, the application can include different kinds of competitions, arranged by for example, teachers for their kindergarten or school classes. These competitions could include competitions about the number of carrots eaten or about most versatile food diet, for example. Users with best scores could be publicly ranked in top ten or top one-hundred within the application.

FIG. 3 depicts another example of the user interface of the application resident on user's mobile terminal (5). At the beginning, the energy levels are high (12).

When the application is in use i.e., user in playing the game, the energy levels of the character start to decrease, and the character becomes unhappy (51) to show the user that it is time to play and feed the character. The character changes and gets happier once the child feeds it either with by taking photographs of vegetables, fruits, nuts or berries, as seen on FIG. 3 (14), or interacts with codes within vegetable section of a store. In this example, user clicks on the icon representing photography (13) and follows through by photographing a paprika in this example as seen on items (14-18). In particular (16) is an animation taking place inside a human body. The stomach and intestines can be seen in caricature format.

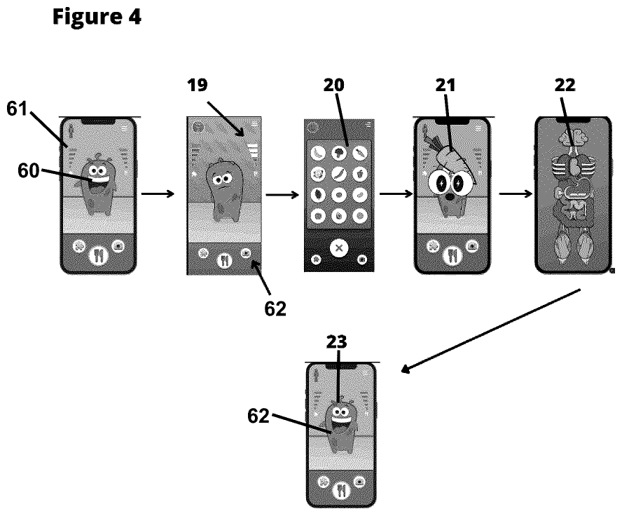

Alternatively, the user can feed the character with virtual vegetables, fruits, nuts or berries in the application, as seen in FIG. 4, items 19-23. The character starts out happy (60) as usual with high energy levels (61). But as the character becomes hungry, they decrease (19). By touching button (62), the child user can access a menu board (20) of possible vegetables, fruits, nuts or berries. As the user chooses various food items, their particular icon shows on the screen (21, 22). In the end, the character is again happy (23), and the energy levels are high again (62).

In another embodiment, the energy levels of the character decrease gradually over time whether the application has been in used or not.

The energy bar (7) (FIG. 2) shows if the character is hungry and needs to be fed with vegetables, fruits, nuts or berries. The energy levels of the bar start to decrease once the application is opened, and the look of the character changes as well. The icon of camera opens the photography function of the application (13). The function sends the photograph (14) to external service (4), via image control part (3). External service (4) provider, after it has recognized the vegetable, fruit or berry, sends information about the photographed vegetable, fruit or berry back to the application. The application responds only to vegetables, fruits, nuts & berries which are defined in the application, and this feature rewards the user by activating the animation related to so called “superpower” of the chosen vegetable, fruit or berry, by combining the photograph information with official health & nutrition claims from external sources and bringing them up in the form of an animation (15), and by opening the minigame or animation inside of a human body (16).

By recognizing vegetables, fruit & veggies the user also gets badges to the home screen of main character, Vegemi. These badges are kept there for the time the parent user has decided in the application settings, letting the parent user decide how often the child user should try to collect a variety of veggies again. After completing function (13), the character looks energized again (18) and the energy baro become(s) high.

The application can provide user and their guardian with information on the photographed or interacted vegetables, fruits, nuts or berries. It can, for example recommend a snack that includes photographed or interacted vegetable, fruit or berry. In alternative embodiment of the invention, the application can provide user or their guardian with recipe of a meal that includes photographed or interacted vegetables, fruits, nuts or berries. In alternative embodiment of the invention, the application can provide user or their guardian with a shopping list for ingredients of a meal that includes photographed or interacted vegetables, fruits, nuts or berries.

FIG. 4 depicts one example of the user interface of the application resident on user's mobile terminal (5).

The character of the application can be fed with virtual vegetables, fruits, nuts & berries as well. The energy lever bar (19) shows when the character is hungry and in need of vegetables, fruits, nuts & berries. In addition to photographing vegetables, fruits, nuts & berries or interacting with materials placed in the vegetable department of the store, the user can feed virtual vegetables, fruits, nuts & berries to the character of the application. In order to do so, the user chooses a vegetable (20) to be fed to character. As a reward, the user will get to watch a shorter version of animation (21) and learns where the chosen fruit, vegetable or berry influences in the human body. The characters energy level recharges and the visual representation of character changes (23).

The application can also learn preferences and likes of the user based on virtual vegetables, fruits, nuts & berries fed to the character in application, and recommend a vegetable, fruit or berry that user likes to feed to the character, but isn't photographing/interacting/eating in real life. The recommendations are sent directly to user, user's guardian, teacher or to combination of them.

The main function of this part is to educate children and their guardians about the impacts of eating healthy and different effects different vegetables, fruits, nuts & berries might have.

By combining nutrition recommendations of public authorities, nutritional information of fruits, vegetables and berries, and harvest season information with the consumption habits of young children, the system is able to generate specific nutritional recommendations to single user based on their possible needs of vitamins, trace elements and fibres.

By combining nutritional recommendations with gamification, we are able to promote healthy nutrition to young children more efficiently, reduce overweight or obesity in children and improve public health.

Several descriptions and illustrations have been presented to aid in understanding the present invention. One with skill in the art will realize that numerous changes and variations may be made without departing from the spirit of the invention. Each of these changes and variations is within the scope of the present invention.

1. A system for gamification of nutritional guidance for children and their guardians comprising:
a first computer software product configured to run on a hand-held mobile device;
a second computer software product configured to run on a remote server; the first computer software product in communication with the second computer software product via a network;
a database stored a storage accessible by the remote server, the database containing dates of harvest seasons of vegetables, fruits, nuts or berries; and health recommendations concerning vegetables, fruits, nuts and berries;
a computer-generated character displayable on the hand-held telephone;
wherein, the second computer software product is configured to access said database upon receiving a photograph or reference to a particular vegetable, fruit or berry a child user has eaten, and return stored nutritional information about that particular vegetable, fruit or berry including recommendations and harvest dates;
wherein, the first or second computer software product causes the computer-generated character to favourably gesture approval to the child user for eating said vegetable, fruit or berry.

2. The system for gamification of nutritional guidance of claim 1, further comprising the first or second computer software product rewarding the child user with a symbolic icon for eating the vegetable, fruit or berry.

3. The system for gamification of nutritional guidance of claim 2, wherein the symbolic icon represents the vegetable, fruit or berry eaten by the child user.

4. The system for gamification of nutritional guidance of claim 1, wherein the computer-generated character represents a friendly intestinal bacterium needing good food in order to increase in energy to promote the child's health.

5. The system for gamification of nutritional guidance of claim 4, wherein an energy bar is displayed on the telephone showing the energy of the computer-generated character, and the computer-generated character displays happiness when energy increases and sadness and fatigue when energy decreases.

6. The system for gamification of nutritional guidance of claim 1, wherein the recommendations are formed utilizing location data of child user.

7. The system for gamification of nutritional guidance of claim 1, wherein the recommendations are formed utilizing the child user's peer group's nutritional history.

8. The system for gamification of nutritional guidance of claim 1, wherein the recommendations are formed utilizing the child user's known allergies.

9. The system for gamification of nutritional guidance of claim 1, wherein the recommendations are formed based on location data of user and promotions of groceries proximate to user.

10. The system for gamification of nutritional guidance of claim 1, wherein the recommendations are formed utilizing user's nutritional goals.

11. The system for gamification of nutritional guidance of claim 1, wherein the recommendations are formed utilizing harvest dates and current time of year.

12. The system for gamification of nutritional guidance of claim 5, wherein the energy bar displays a decrease in energy with time if the computer-generated character is not fed vegetables, fruits, nuts or berries.

13. A method executable on a computer that encourages a child user to eat more vegetables, fruits, nuts or berries comprising:
placing an animated character on a computer screen representing a friendly intestinal bacterium, the animated character having an energy level represented by a plurality of energy bars;
decreasing said energy level with time if the animated character is not fed at least one of a vegetable, a fruit, a type of nut, or a type of berry, and causing the animated character to display unhappiness.
presenting on the computer screen a selection of icons representing vegetables, fruits, nuts and berries;
allowing the child user to choose one of said vegetables, fruits, nuts or berries in said selection to feed the animated character;
enlarging at least one feature of the animated character upon being feed the chosen vegetable, fruit, nut or berry;
displaying an animation of digestion of the chosen vegetable, fruit, nut or berry in a caricature of a human body;
increasing the energy level displayed by the plurality of energy bars, and having the animated character to display happiness.

14. The method of claim 13, wherein the at least one feature enlarged is eyes of the animated character.
